(19) United States
(12) Patent Application Publication
(10) Pub. No.: US 2009/0222064 A1
(43) Pub. Date: Sep. 3, 2009

(54) Autonomous Autoprogram Cochlear Implant

(75) Inventors: Michael A. Faltys, Northridge, CA (US); Timothy J. Starkweather, Simi Valley, CA (US); Anthony K. Arnold, Valencia, CA (US)

Correspondence Address: Wong Cabello Lutsch Rutherford & Brucculeri LLP, 20333 Tomball Pkwy, Suite 600, Houston, TX, 77070, US

(73) Assignee: Advanced Bionics, LLC, Valencia, CA (US)

(21) Appl. No.: 12/437,861

(22) Filed: May 8, 2009

Related U.S. Application Data
(62) Division of application No. 11/178,054, filed on Jul. 8, 2005.

(51) Int. Cl. A61F 11/04; A61N 1/36

(52) U.S. Cl. 607/57

(57) ABSTRACT

Disclosed is a cochlear stimulation system having patient parameters that reside in memory of an internal portion of the system. Different external systems define how the cochlear stimulation system processes a received acoustic signal and uses patient information uploaded from an implant to parameterize system processing. The external system uses external and internal processing capability to convert acoustic signals to electrical stimulus most appropriate for the patient. Because the patient parameters reside internally, the external portion of the system can be replaced to provide an external replacement processor and potentially offer the patient an new type of program without having to re-program the cochlear stimulation system.

### CROSS-REFERENCE TO RELATED APPLICATIONS

This is a divisional application of U.S. patent application Ser. No. 11/178,054, filed Jul. 8, 2005, to which priority is claimed and which is incorporated herein by reference.

### TECHNICAL FIELD

This disclosure relates to systems and methods for stimulating the cochlea, and more particularly to systems and methods for fitting a cochlear implant to a user.

### BACKGROUND

Prior to the past several decades, scientists generally believed that it was impossible to restore hearing to the deaf. However, scientists have had increasing success in restoring normal hearing to the deaf through electrical stimulation of the auditory nerve. The initial attempts to restore hearing were not very successful, as patients were unable to understand speech. However, as scientists developed different techniques for delivering electrical stimuli to the auditory nerve, the auditory sensations elicited by electrical stimulation gradually came closer to sounding more like normal speech. The electrical stimulation is implemented through a prosthetic device, called a cochlear stimulation system, that interacts with the inner ear to restore partial hearing to profoundly deaf people.

A cochlear stimulation system generally includes an internal portion that includes an electrode array that is inserted in a cochlear duct, usually the scala tympani. One or more electrodes of the array selectively stimulate different auditory nerves at different places in the cochlea based on the pitch of a received sound signal. The internal portion interacts with an external portion that includes a speech processor that processes converted acoustic signals in accordance with a selected speech processing strategy to generate appropriate control signals for controlling the electrode array.

In order for the patient to properly perceive sounds with the cochlear stimulation system, the system must be “fitted” or “tuned” to accommodate the electrode array's particular placement in the patient's cochlea. Such a fitting method includes a pitch ranking and channel allocation process. Pursuant to this process, the electrodes of the electrode array are ranked based on their pitch. The speech processor then assigns certain frequency bands to each electrode of the array such that each electrode is associated with a particular channel that represents a frequency or range of frequencies.

The fitting process can be time consuming and tedious for both the patient and for the clinician that is performing the fitting process. In view of the foregoing, there is a need for a cochlear stimulation system that minimizes the need to repeat the fitting process for a patient.

### SUMMARY

Disclosed is a cochlear stimulation system having patient parameters that reside in memory of an internal portion of the system. Different external systems define how the cochlear stimulation system processes a received acoustic signal and uses the patient information uploaded from the implant to parameterize system processing. The external system uses external and internal processing capability to convert acoustic signals to electrical stimulus most appropriate for the patient. Because the patient parameters reside internally, the external portion of the system can be replaced to provide an external replacement processor and potentially offer the patient a new type of program without having to re-program the cochlear stimulation system. Some programs may require that patient-specific data to change, other programs will allow the patient to just attach an unprogrammed external portion.

In one aspect, a cochlear stimulation system comprises an external portion and an internal portion. The external portion includes an acoustic transducer for sensing acoustic signals and converting them to electrical signals. The internal portion includes a multi-electrode array having a first plurality of electrodes configured for placement in first cochlear duct of a patient, programmable memory, and a cochlear stimulation program residing in the programmable memory. The cochlear stimulation program includes data that defines sound processing and corresponding cochlear stimulation for the system.

In another aspect, a method of implementing a program for a cochlear stimulation system, comprises implanting an internal portion of a cochlear stimulation system under the skin of a patient, the internal portion including at least one cochlear stimulation program; attaching an external portion of the cochlear stimulation system to the patient; and uploading a first cochlear stimulation program to the external portion from the internal portion.

In another aspect, a cochlear stimulation system comprises an internal portion implantable under the skin of a patient. The internal portion includes a multi-electrode array having a first plurality of electrodes configured for placement in first cochlear duct of a patient and a cochlear stimulation program including data that defines sound processing and corresponding cochlear stimulation for the system. The internal portion is configured to upload the cochlear stimulation program to an external portion of the cochlear stimulation system.

The details of one or more embodiments are set forth in the accompanying drawings and the description below. Other features and advantages will be apparent from the description and drawings, and from the claims.

### DESCRIPTION OF DRAWINGS

The features and advantages will be more apparent from the following more particular description thereof, presented in conjunction with the following drawings, wherein:

FIG. 1 shows a cochlear implant system capable of providing high rate pulsatile electrical stimuli to the cochlea of a patient.

FIG. 2 shows a partial functional block diagram of the cochlear stimulation system.

FIG. 3 schematically shows an external portion and an internal portion of the cochlear stimulation system, the internal portion including one or more cochlear stimulation programs.

Like reference symbols in the various drawings indicate like elements.

### DETAILED DESCRIPTION

Disclosed are devices and methods for matching information between cochlear implants in two ears of a patient. It will be helpful to first provide an overview of the structure and functionality of an exemplary cochlear implant system. This overview is provided below in connection with the description of FIG. 3. It should be appreciated that the following description is exemplary and that the device and methods described herein can be used with other types and other configurations of cochlear implant systems.

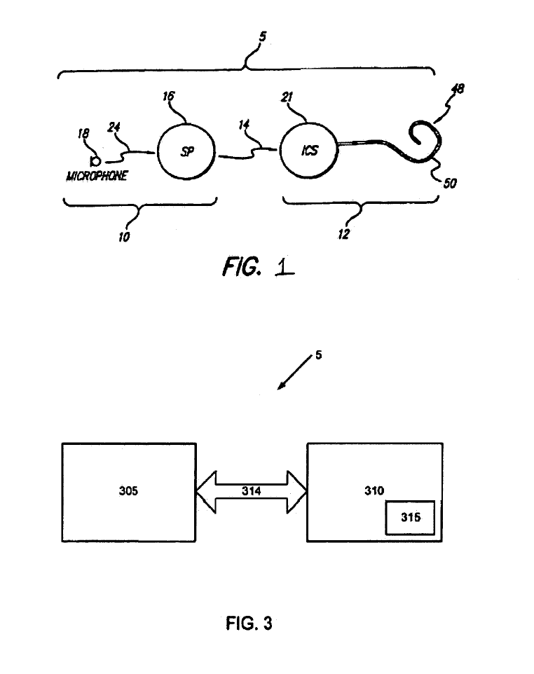

FIG. 1 shows a cochlear stimulation system 5 that includes a speech processor portion 10 and a cochlear stimulation portion 12. The speech processor portion 10 includes a speech processor (SP) 16 and a microphone 18. The microphone 18 may be connected directly to the SP 16 or coupled to the SP 16 through an appropriate communication link 24.

The cochlear stimulation portion 12 includes an implantable cochlear stimulator (ICS) 21 and an electrode array 48. The electrode array 48 is adapted to be inserted within the cochlea of a patient. The array 48 includes a plurality of electrodes 50, e.g., sixteen electrodes, spaced along the array length and which electrodes are selectively connected to the ICS 21. The electrode array 48 may be substantially as shown and described in U.S. Pat. No. 4,819,647 or 6,129,753, both patents incorporated herein by reference.

The ICS 21 and the SP 16 are linked together electronically through a suitable data or communications link 14. The data link 14 can be a transcutaneous (through the skin) data link that allows power and control signals to be sent from the SP 16 to the ICS 21. In some embodiments, data and status signals may also be sent from the ICS 21 to the SP 16.

At least certain portions of the cochlear stimulation system 5 can be included within an implantable portion that is implanted beneath the patient's skin, while other portions of the cochlear stimulation system 5 can remain in an external portion of the system. In general, at least the microphone 18 and associated analog front end (AFE) circuitry (described below) are part of the external portion of the system, and at least the ICS 21 and the electrode array 48 are part of the implantable portion of the system. Moreover, certain portions of the external portion of the cochlear stimulation system 5 can be contained in a behind the ear (BTE) unit that is positioned at or near the patient's ear. For example, the BTE unit can include the SP 16 and a battery module, which are coupled to a corresponding ICS 21 and an electrode array 48.

As used herein, the term “external” means not implanted under the skin or residing within the inner ear. However, the term “external” can also mean residing within the outer ear, residing within the ear canal or being located within the middle ear.

As mentioned above, in order for the patient to properly perceive sounds with the cochlear stimulation system 5, the system must be fitted or tuned to accommodate the electrode array's particular placement in the patient's cochlea. Such a fitting method generally requires a clinician to spend a period of time with the patient tuning the system to the patient's particular requirements. The result of the fitting process is at least one “program” (referred to herein as a cochlear stimulation program) that is particularly suited for the patient. The cochlear stimulation program includes various parameters that define how the cochlear stimulation system processes a received acoustic signal, including how the system converts the acoustic signal into a digital signal and maps components of the digital signal to the electrodes in the electrode array. It should be appreciated that a particular patient can have multiple cochlear stimulation programs that vary based upon a particular acoustic environment of the patient.

The cochlear stimulation program generally includes a mechanism for transforming acoustic signals to stimulus that executes on internal and external hardware. The program is parameterized through “strategy parameters” and “stimulation parameters” that are adjusted to each patient ear. The strategy parameters define how the speech processor transforms a received acoustic signal into to a stimulation waveform, while patient-specific stimulation parameters determine acoustic processing options of the external processor and define how the stimulation current is mapped to the electrodes in the array as a function of information contained within the sensed acoustic signal. Electronic circuitry within the ICS 21 allows a specified stimulation current to be applied to selected pairs or groups of the individual electrodes included within the electrode array 48 in accordance with a specified stimulation pattern defined by the SP 16.

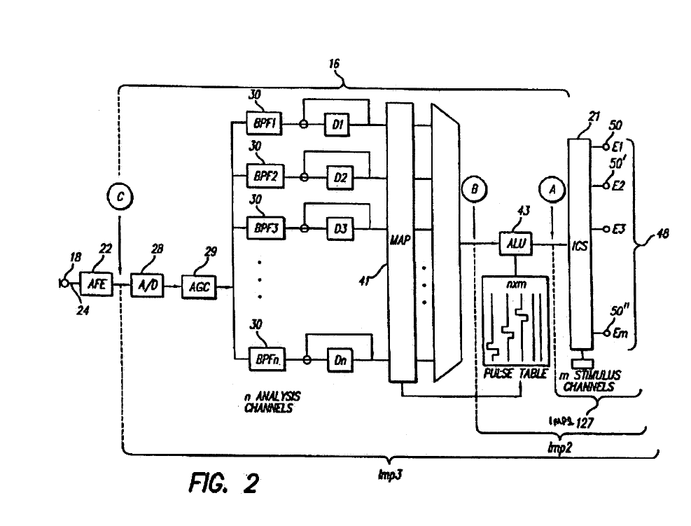

FIG. 2 shows a partial block diagram of one embodiment of a cochlear implant system capable of providing a high pulsatile stimulation pattern. FIG. 2 depicts the functions that are carried out by the SP 16 and the ICS 21. The process generally begins when the microphone 18 is exposed to sound waves. The microphone 18 senses the sound waves and converts such sound waves to corresponding electrical signals and thus functions as an acoustic transducer. The electrical signals are sent to the SP 16 over a suitable electrical or other link 24. The SP 16 processes these converted acoustic signals in accordance with a selected speech processing strategy to generate appropriate control signals for controlling the ICS 21. Different speech processing strategies require different external software and sometimes different external hardware. It is conceivable that each different sound coding strategy will require a different external processor rather than downloading different code into a generic external processor. It is the task of the external processor to understand how to use the patient (ear) specific data stored in the implant in the context of the implemented program. The external software/hardware that performs this function is configured at the factory.

The speech processing strategy was developed during the fitting process described above. The control signals specify or define the polarity, magnitude, location (which electrode pair or electrode group receive the stimulation current), and timing (when the stimulation current is applied to the electrode pair) of the stimulation current that is generated by the ICS. Such control signals thus combine to produce a desired spatio-temporal pattern of electrical stimuli in accordance with a desired speech processing strategy.

A speech processing strategy is used, among other reasons, to condition the magnitude and polarity of the stimulation current applied to the implanted electrodes of the electrode array 48. Such speech processing strategy involves defining a pattern of stimulation waveforms that are to be applied to the electrodes as controlled electrical currents.

It should be appreciated that the functions shown in FIG. 2 (dividing the incoming signal into frequency bands and independently processing each band) are representative of just one type of signal processing strategy that may be employed. Other signal processing strategies could just as easily be used to process the incoming acoustical signal. A description of the functional block diagram of the cochlear implant shown in FIG. 2 is found in U.S. Pat. No. 6,219,580, incorporated herein by reference. The system and method described herein may be used with other cochlear systems other than the system shown in FIG. 2, which system is not intended to be limiting.

The cochlear implant functionally shown in FIG. 2 provides n analysis channels that may be mapped to one or more stimulus channels. That is, after the incoming sound signal is received through the microphone 18 and the analog front end circuitry (AFE) 22, the signal can be digitized in an analog to digital (A/D) converter 28 and then subjected to appropriate gain control (which may include compression) in an automatic gain control (AGC) unit 29. After appropriate gain control, the signal can be divided into n analysis channels 30, each of which includes at least one bandpass filter, BPFn, centered at a selected frequency. The signal present in each analysis channel 30 is processed as described more fully in the U.S. Pat. No. 6,219,580, or as is appropriate, using other signal processing techniques. The signals from each analysis channel may then be mapped, using mapping function 41, so that an appropriate stimulus current of a desired amplitude and timing may be applied through a selected stimulus channel to stimulate the auditory nerve.

The exemplary system of FIG. 2 provides a plurality of analysis channels, n, wherein the incoming signal is analyzed. The information contained in these n analysis channels is then appropriately processed, compressed and mapped in order to control the actual stimulus patterns that are applied to the user by the ICS 21 and its associated electrode array 48.

The electrode array 48 includes a plurality of electrode contacts 50, 50′, 50″ and labeled as, E1, E2, . . . Em, respectively, which are connected through appropriate conductors to respective current generators or pulse generators within the ICS. Through these plurality of electrode contacts, a plurality of stimulus channels 127, e.g., m stimulus channels, may exist through which individual electrical stimuli can be applied at m different stimulation sites within the patient's cochlea or other tissue stimulation site.

The cochlear stimulation program is typically stored in volatile memory located in the external portion of the cochlear stimulation system. Storage of the cochlear stimulation program in the external portion presents drawbacks. For example, if the external portion of the system has to be replaced, such as if the patient loses or damages the external portion, the fitting process has to be re-performed for the new external portion. This can be undesirable, as it requires the patient to go through the time consuming fitting process all over again.

There is now described an embodiment of the cochlear stimulation system wherein the cochlear stimulation program is stored in the implantable portion of the system. FIG. 3 shows a schematic representation of the cochlear stimulation system 5, which includes the components described previously with reference to FIG. 2, including the speech processor 16, which can reside in an external portion of the system. As mentioned, the cochlear stimulation system includes an external portion 305 and an internal portion 310 that are communicatively linked via a communications link 314.

The internal portion 310 includes programmable memory 315 that can be used to store data, such as strategy parameters and the stimulation parameters of one or more cochlear stimulation programs. The data stored in the programmable memory 315 can be communicated to, or reprogrammed by, the external portion 305 through one-way or bi-directional communication. The programmable memory can be volatile or non-volatile memory. Non-volatile memory advantageously eliminates the need for re-loading of the cochlear stimulation programs upon loss of power to the system.

It should be appreciated that the programmable memory is not limited to storing a single cochlear stimulation program. Multiple cochlear stimulation programs can reside in the programmable memory 315. In this regard, an external controller can be configured to permit the patient to select a desired the cochlear stimulation program on the fly. For example, the programmable memory 315 can include a first cochlear stimulation program that is particularly suited for relatively loud environments and a second cochlear stimulation program that is used for more quiet environments. Depending on the environment, the patient can upload the appropriate cochlear stimulation program from the internal portion to the external portion of the cochlear stimulation system.

The cochlear stimulation program(s) are preferably downloaded to the programmable memory 315 of the internal portion 310 via the communication link 314 shortly after the fitting process. With the cochlear stimulation program(s) residing in the programmable memory 315, the speech processor 16 can extract the data from the cochlear stimulation program to the external portion 305. This permits the external portion to be modified or replaced without losing the cochlear stimulation program(s) and without having to re-program the cochlear stimulation system.

In an exemplary method of establishing or implementing a program for the cochlear stimulation system, the cochlear stimulation system is first coupled to the patient. This includes implanting the internal portion of a cochlear stimulation system under the skin of a patient, such as by implanting the multi-electrode array in the cochlea. The external portion of the cochlear stimulation system is also coupled to the patient and a communication link is established between the internal portion and the external portion.

Pursuant to a fitting or tuning process (see, e.g., U.S. Pat. No. 6,289,247, incorporated herein by reference, for an example of one type of fitting or tuning process) one or more cochlear stimulation programs are created for the patient. As mentioned, the cochlear stimulation program includes various parameters that define how the cochlear stimulation system processes a received acoustic signal, including how the system converts the acoustic signal into a digital signal and maps components of the digital signal to the electrodes in the electrode array. The one or more cochlear stimulation programs are then loaded into the programmable memory of the internal portion.

This permits the patient or a clinician to upload the cochlear stimulation program (or a portion thereof) to the external portion of the cochlear stimulation system on an as-needed basis. For example, the cochlear stimulation program can be uploaded from the internal portion to the external portion when the external portion is replaced. A new cochlear stimulation program can be uploaded to the external portion from the internal portion where the user desires to use a different version of the program, such as where the audio environment changes. Advantageously, the external portion can be exchanged or replaced without having to re-tune the cochlear stimulation system.

A number of embodiments have been described. Nevertheless, it will be understood that various modifications may be made without departing from the spirit and scope of the claims. Accordingly, other embodiments are within the scope of the following claims.

What is claimed is:

1. A method for operating a cochlear stimulation system worn by a patient comprising an external portion and an internal portion, comprising:
performing a fitting procedure on the patient to determine a first patient specific cochlear stimulation program for use by the external portion in processing acoustic signals sensed at the external portion;
storing the first patient specific cochlear stimulation program in the internal portion internal to the patient;
uploading the stored first patient specific cochlear stimulation program from the internal portion to the external portion external to the patient; and
processing sensed acoustic signals at the external portion using the uploaded first patient specific cochlear stimulation program.

2. The method of claim 1, wherein determining a first patient specific cochlear stimulation program comprises a fitting procedure.

3. The method of claim 1, wherein the processed acoustic signals are used to stimulate electrodes coupled to the internal portion.

4. The method of claim 3, wherein the electrodes are supported by an electrode array.

5. The method of claim 4, wherein the electrode array is implanted on cochlea of the patient.

6. The method of claim 1, wherein the first patient specific cochlear stimulation program comprises patient specific strategy parameters and patient specific stimulation parameters.

7. The method of claim 6, wherein the processing comprises:
transforming sensed acoustic signals to a stimulation waveform based on the strategy parameters; and
mapping stimulation waveform components to electrodes coupled to the internal portion based on patient specific stimulation parameters.

8. The method of claim 7, further comprising transmitting control signals to the internal portion, the control signals being a function of a result of the mapping of stimulation waveform components to electrodes.

9. The method of claim 8, further comprising using the control signals to control polarities, magnitudes, locations and timings of stimulation currents applied to the electrodes.

10. The method of claim 1, further comprising:
replacing the external portion while leaving the internal portion in the patient; and
uploading to the external portion from the internal portion a second patient specific cochlear stimulation program for use by the external portion in processing acoustic signals sensed at the external portion.

11. A method for operating a cochlear stimulation system worn by a patient comprising an external portion and an internal portion, comprising:
performing a fitting procedure on the patient to determine a patient specific cochlear stimulation program for use by the external portion in processing acoustic signals sensed at the external portion;
storing the patient specific cochlear stimulation program in the internal portion internal to the patient;
uploading the stored patient specific cochlear stimulation program from the internal portion to the external portion external to the patient;
processing sensed acoustic signals at the external portion using the uploaded patient specific cochlear stimulation program; and
transmitting the processed acoustic signals from the external portion to the internal portion.

12. The method of claim 11, wherein the processed acoustic signals are used to stimulate electrodes coupled to the internal portion.

13. The method of claim 12, wherein the electrodes are supported by an electrode array.

14. The method of claim 13, wherein the electrode array is implanted on cochlea of the patient.

15. The method of claim 11, wherein the patient specific cochlear stimulation program comprises patient specific strategy parameters and patient specific stimulation parameters.

16. The method of claim 15, wherein the processing comprises:
transforming sensed acoustic signals to a stimulation waveform based on the strategy parameters; and
mapping stimulation waveform components to electrodes coupled to the internal portion based on patient specific stimulation parameters.

17. The method of claim 16, further comprising transmitting control signals to the internal portion, the control signals being a function of a result of the mapping of stimulation waveform components to electrodes.

18. The method of claim 17, further comprising using the control signals to control polarities, magnitudes, locations and timings of stimulation currents applied to the electrodes.
